(19) United States
(12) Patent Application Publication
(10) Pub. No.: US 2014/0296281 A1
(43) Pub. Date: Oct. 2, 2014

(54) METHODS FOR SCREENING A BRCA1 LOSS-OF-FUNCTION IN A SUBJECT SUFFERING FROM A CANCER

(71) Applicant: INSERM (INSTITUT NATIONAL DE LA SANTE ET DE LA RECHERCHE MEDICALE), Paris (FR)

(72) Inventors: Anne Gompel, Paris (FR); Patricia Forgez, Paris (FR)

(21) Appl. No.: 14/360,416

(22) PCT Filed: Nov. 26, 2012
(86) PCT No.: PCT/EP2012/073559

§ 371 Date: May 23, 2014

(30) Foreign Application Priority Data
Nov. 25, 2011 (EP) 11306563.5

(51) Int. Cl. G01N 33/68

(52) U.S. Cl. CPC G01N 33/6872
USPC 514/296; 435/7.1

(57) ABSTRACT

The present invention relates to a method for screening a BRCA1 loss-of-function in a subject comprising the step consisting of determining the level of serine 211 phosphorylated glucocorticoid receptor (GR P-Ser211) expression in a tissue sample obtained from said subject.

### FIELD OF THE INVENTION

The present invention relates to methods and kits of screening a BRCA1 loss-of-function in a subject suffering from a cancer.

### BACKGROUND OF THE INVENTION

Breast cancer is the leading cause of cancer-related death in women world-wide. In the recent years, approximately 200,000 and 421,000 annual new cases of breast cancer were registered in the United States and in Europe, respectively (Jemal, Siegel et al.); http://www.invs.sante.fr/applications/cancers/projections2010/.)

Inherited germline mutations in the BRCA1 tumor suppressor gene confer a genetic predisposition to breast and ovarian cancers with increased risk from 80 and 40%, respectively (Easton, Ford et al. 1995; Antoniou, Pharoah et al. 2003). BRCA1 is involved in the maintenance of genome stability through its function in DNA repair, and in transcriptional regulation. BRCA1 tumour suppression is associated with tumours occurring into hormone-responsive tissues (breast and ovary) (Mild, Swensen et al. 1994; Ford, Easton et al. 1998; Boulton 2006). Ninety percent of breast cancers arising in women with BRCA1 germline mutations are of the“triple negative” type devoided of hormone receptor expression (ER and PR) and HER2 amplification.

Studies of the pathology of familial-BRCA1 breast tumours have identified distinct features that these tumours share with a subset of sporadic tumours, indicating a common or similar aetiology. Said cancers are called “BRCAness cancers”. Evidence is indeed accumulating that silencing of this gene, or dysfunction of other genes acting in similar biochemical pathways, is important in the pathogenesis of a significant proportion of sporadic, non-familial cancers. The routine identification of this class of tumours presents considerable challenges: the lack of evidence of a unifying pathological phenotype, the many components of the pathways and numerous potential mechanisms of inactivation.

The link between BRCA1 and steroid hormones, has been investigated during the last decades. Most of the studies have looked at its interaction with estradiol and progesterone. Among the hormones implicated in breast cancer progression, less importance has been devoted so far to glucocorticoids. A growing body of research and clinical studies suggest that stress also contributes to cancer emergence and progression (Lillberg, Verkasalo et al. 2003; Reiche, Nunes et al. 2004). The only link known so far between BRCA1 and glucocorticoids is that hydrocortisone was shown to repress BRCA1 expression in normal and malignant mouse mammary cell lines breast cells (L. Antonova 2008).

### SUMMARY OF THE INVENTION

The present invention relates to a method for screening a BRCA1 loss-of-function in a subject comprising the step consisting of determining the level of serine 211 phosphorylated glucocorticoid receptor (GR P-Ser211) expression in a tissue sample obtained from said subject.

### DETAILED DESCRIPTION OF THE INVENTION

The inventors observed a loss of expression of GR P-Ser211 in the luminal and myoepithelial cells of normal breast tissues from BRCA1 mutated subjects compared to non mutated subjects. Furthermore a drastic loss of GR P-Ser211 expression was also observed in triple negative breast cancer from mutation carriers compared to sporadic triple negative tumours. The inventors further demonstrate that BRCA1 is crucial for GR signalling pathway through its implication in GR phosphorylation.

Accordingly, the present invention relates to a method for screening a BRCA1 loss-of-function in a subject comprising the step consisting of determining the level of serine 211 phosphorylated glucocorticoid receptor (GR P-Ser211) expression in a tissue sample obtained from said subject.

In a particular embodiment, the subject may be apparently healthy, i.e. without signs of breast or ovarian cancer. In another particular embodiment the subject may suffer from a cancer. Typically said cancer is a breast cancer an ovarian cancer or a non small cell lung cancer or a head/neck cancer. Preferably, said subject suffers from a triple negative breast cancer. “Triple negative” breast cancer, means that said breast cancer lacks receptors for the hormones estrogen (ER-negative) and progesterone (PR-negative), and for the protein HER2.

The term “tissue sample” refers to a sample obtained from a tissue wherein a cancer occurs (i.e tumor tissue sample), or from a tissue wherein there is a high probability that a cancer will occur. Typically, the tissue sample results from a biopsy and then after is fixed and paraffin embedded.

“BRCA1” has its general meaning in the art and refers to the DNA, mRNA, or translated protein of BRCA1 gene. BRCA1 is also known as IRIS; PSCP; BRCAI; BRCC1; PNCA4; RNF53; BROVCA1; and PPP1R53.

The term “BRCA1 loss-of-function” relates to any dysfunction observed for BRCA1. Non-functioning of BRCA1 may be due to germline mutations in the BRCA1 gene as observed in familial-BRCA1 breast tumours. Non-functioning of BRCA1 may also be due to acquired loss of function in BRCA1 as observed in sporadic cancers. “Sporadic cancer” refers to cancer due to mutations or tumors caused by environmental or other factors that does not include those highly penetrant mutations in breast cancer predisposing genes inherited from either or both parents of the individual. Sporadic tumors may include low-penetrance genes inherited from either or both parents of the individual. Sporadic tumors may also arise as a result of DNA methylation of the BRCA1 gene or promoter or other epigenetic mechanisms.

The term “glucocorticoid receptor”, also known as NR3C1 (nuclear receptor subfamily 3, group C, member 1), has its general meaning in the art and refers to a ligand-activated transcription factor that binds with high affinity to Cortisol and other glucocorticoids. The glucocorticoid receptor may be phosphorylated at multiple serine residues and especially at serines 203, 211 and 226 (S203, S211, and S226). Accordingly the term “serine 211 phosphorylated glucocorticoid receptor” or “GR P-Ser211” refers to the glucocorticoid receptor phosphorylated at serine 211.

Determining the level of GR P-Ser211 level may be determined by any well known method in the art. Typically, such methods comprise contacting the tissue sample with a binding partner capable of selectively interacting with GR P-Ser211. The binding partner may be polyclonal antibody or monoclonal antibody, an antibody fragment, synthetic antibodies, or other protein-specific agents such as nucleic acid or peptide aptamers. The preferred method according to the present invention is immunohistochemistry. Accordingly, monoclonal antibodies specific for GR P-Ser211 are preferred for this purpose due to specificity and availability, but polyclonal antibodies as described in the Example could be suitable. Several antibodies have been described in the prior art. For example the document US 2004/0248210 disclose several antibodies specific to GR P-Ser211. Many antibodies are also commercially available such as described in the EXAMPLE. In addition, antibodies may be easily generated using techniques well-known to those skilled in the art. The use of antibodies to identify proteins of interest in the cells of a tissue, referred to as immunohistochemistry (IHC), is well established. See for exemple “Principles and practice of immunoassays” 1991, C. P. Price and D. J. Neoman (eds) Stockton Press, N.Y. For the detection of the antibody that makes the presence of the protein of interest detectable by microscopy or an automated analysis system, GR P-Ser211 antibodies may be tagged directly with detectable labels such as enzymes, chromogens or fluorescent probes or indirectly detected with a secondary antibody conjugated with detectable labels. The preferred staining method according to the present invention uses a secondary antibody coupled to an amplification system (to intensify staining signal) and enzymatic molecules. Such coupled secondary antibodies are commercially available, e.g. from Dako, EnVision system. Counterstaining may be used, e.g. H&E, DAPI, Hoechst. Other staining methods may be accomplished using any suitable method or system as would be apparent to one of skill in the art, including automated, semi-automated or manual systems.

In a particular embodiment, the method comprises quantification of the level of GR P-Ser211 expression. Typically, the level of GR P-Ser211 expression is determined by level of intensity of GR P-Ser211 staining in the nuclei of tumour cells (e.g. nuclei of the myoepithelial and luminal cells for breast cancer) present in the whole tissue sample obtained from the subject. Alternatively, the percentage of positive cells for GR P-Ser211 expression may also be determined. In another embodiment the Allen score which is a composite of the percentage of cells that stained and the intensity of their staining may be also determined.

In a particular embodiment, the method of the invention comprises the steps consisting in i) providing one or more immunostained slices of tissue section obtained by an automated slide-staining system by using a binding partner capable of selectively interacting with GR P-Ser211 (e.g. an antibody as above descried), ii) proceeding to digitalisation of the slides of step a. by high resolution scan capture, iii) detecting the slice of tissue section on the digital picture iv) providing a size reference grid with uniformly distributed units having a same surface, said grid being adapted to the size of the tissue section to be analyzed, and v) detecting, quantifying and measuring intensity of stained cells in each unit whereby the number or the density of cells stained of each unit is assessed.

Typically the Allen score which is a composite of the percentage of cells that stained and the intensity of their staining is also determined in a last step.

For example an IHC automate such as BenchMark® XT allowing automatic stained slide preparation may be used for implementing the immunohistochemical staining step. Digitalisation of the slides may be typically made by scan capture, for example with a high resolution Hamamatsu NanoZoomer® 2.0-HT scanner allowing scanning standard-size (26 mm×76 mm) slides. This scanner provides high definition digital pictures (×20: 0.46 μm/pixel is preferred) and (×40: 0.23 μm/pixel).

In a particular embodiment, the method of the invention further comprises the step of comparing the level of GR P-Ser211 expression determined in the tissue sample with a reference value, wherein detecting differential in the level of expression determined in the tissue sample and the reference value is indicative that the subject has a BRCA1 loss-of-function.

A reference value can be a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. The threshold value has to be determined in order to obtained the optimal sentivity and specificity according to the function of the test and the benefice/risk balance (clinical consequences of false positive and false negative). Typically, the optimal sentivity and specificity (and so the threshold value) can be determined using a Receiver Operating Characteristic (ROC) curve based on experimental data. Preferably, the person skilled in the art may compare the expression levels of GR P-Ser211 expression obtained according to the method of the invention with a defined threshold value. In one embodiment of the present invention, the threshold value is derived from the level GR P-Ser211 expression in a control sample derived from one or more subjects who are substantially healthy (i.e. having no cancer). In one embodiment of the present invention, the threshold value may also be derived from the level GR P-Ser211 expression in a control sample derived from one or more subjects who have cancer without BRCA1 loss-of-function. Furthermore, retrospective measurement of level of GR P-Ser211 expression levels in properly banked historical subject samples may be used in establishing these threshold values. Typically, the levels of GR P-Ser211 expression in a cancer subject having a BRCA1 loss-of-function is deemed to be lower than the reference value obtained from cancer subjects or healthy subjects without BRCA1 loss-of-function. Indeed, the inventors have established that GR P-Ser211 staining is strong and concerns 50% or more of the cells in breast cancer without BRCA1 loss-of-function, whereas the GR P-Ser211 staining is significantly less expressed in breast cancers associated with BRCA1 mutations. In other words, subjects that present at least 50% of epithelial cells positive for GR P-Ser211 have high risk to have a BRCA1 loss-of-function.

The method of the invention is particularly suitable for the management of subjects who are apparently healthy but having a history of cancer, especially breast cancer or ovarian cancer in their family. For those subjects the method of the invention offers a reliable tool for monitoring the risk of having a cancer. The method of the invention is also particularly suitable for the management of subjects having cancer and having a history of cancer in their family. For example, only 20% of women with a history of breast cancer who undergo comprehensive mutation analysis have identifiable BRCA1 mutations. This is a costly approach since each comprehensive analysis costs about $3000, with a corresponding cost of $15,000 to identify a single mutation carrier. By the method of the invention as a pre-screen, comprehensive mutation analysis could then be targeted to women whose a BRCA1 loss-of-function could be detected by the method of the invention.

In order to provide targeted treatments, it will be crucial to identify subjects with tumours that have BRCA1 loss of function at the time of diagnosis in order to provide targeted treatment. For example, current comprehensive BRCA1 testing has a routine turn-around time of 3-4 weeks which may be too long for a timely diagnosis of inherited loss of BRCA1 function. Furthermore, gene testing will not identify somatic (acquired) loss of BRCA1 function. Therefore the method of the present invention is highly useful for identifying tumors with BRCA1 loss of function in a timely manner in order to use this information to guide targeted treatments. The method of the invention indeed provides an opportunity for targeted treatment of germline-mutated BRCA1 breast cancers and the 15-30% of sporadic breast cancer with somatic inactivation of BRCA1 by mechanisms including DNA methylation or other epigenetic events. Breast tumors with BRCA1 loss-of-function are particularly eligible for treatment with to PARP [Poly(ADP-ribose) polymerase] inhibitors due to underlying defect in DNA double-stranded break repair.

Accordingly, the present invention also related to a PARP inhibitor for use in the treatment of subjects suffering from a cancer for which a BRCA1 loss-of-function has been determined by the method of the present invention.

PARP inhibitors are well known in the art (Gianluca Papeo, Barbara Forte, Paolo Orsini, Claudia Perrera, Helena Posteri, Alessandra Scolaro, Alessia Montagnoli. Poly(ADP-ribose) polymerase inhibition in cancer therapy: are we close to maturity? Expert Opinion on Therapeutic Patents Oct 2009, Vol. 19, No. 10, Pages 1377-1400: 1377-1400). Examples of PARP inhibitors include but are not limited to 4-Amino-1,8-naphthalimide (ANI), ABT-888 (Abbot Labs), KU59436 (AstraZeneca), AZD2281/Olaparib (Astra-Zeneca), AGOI4699 (Pfizer), BSI-201 (BiPar), INO1001 (Genentech), and GPI 21016 (MGI Pharma).

The present invention also relates to kits for performing the method according to the invention comprising means for determining the level of GR P-Ser211 expression.

According to the invention, the kits of the invention may comprise an anti-GR P-Ser211 antibody; and another molecule coupled with a signalling system which binds to said GR P-Ser211 antibody.

Typically, the antibodies or combination of antibodies are in the form of solutions ready for use. In one embodiment, the kit comprises containers with the solutions ready for use. Any other forms are encompassed by the present invention and the man skilled in the art can routinely adapt the form to the use in immunohistochemistry.

The invention will be further illustrated by the following figures and examples. However, these examples and figures should not be interpreted in any way as limiting the scope of the present invention.

### FIGURES

FIG. 1: Total and P-Ser211 Glucocorticoid receptor expressions in normal and cancerous breast tissues.

(A, B), Immunolabelling of total GR in luminal (black arrows) and myoepithelial cells (red arrows) in alveolar/lobular structures of 23 BRCA1+/+ (A) and 20 BRCA1+/− (B) normal human breast tissues (original magnification ×400). (C, D), Immunolabelling of total GR P-Ser211 in luminal (black arrows) and myoepithelial cells in alveolar/lobular structures of BRCA1+/+ (C,E) and BRCA1+/− (D,F) normal human breast tissues (original magnification ×400), Immunodetection of total GR P-Ser211 of 14 BRCA1+/+ (G) and 13 BRCA1−/− (H) triple negative (TN) human breast tissues (original magnification ×400). (I), Representation of the percentage of positive cells for GR P-Ser211 and mean intensity score of GR P-Ser211 staining in tumour cells from BRCA1+/+ and BRCA1−/− triple negative patients. (***p<0.0001).

### EXAMPLE

### Material & Methods

Reagents and Steroids

Dexamethasone (DEX), insulin, Epidermal Growth Factor (EGF), transferrin, choleratoxin, cortisol, hyaluronidase, thymidine, mevinolin, ribonuclease A, were purchased from Sigma-Aldrich (St Quentin Fallavier, France). The glucocorticoid receptor antagonist ORG34116 (AG) was kindly provided by Dr H J Kloosterboer ex-Organon (Oss, The Netherlands).

Immunohistochemistry

Breast samples were obtained from 23 normal BRCA1+/+ women (mean age of 34.7±10.7 years), from 20 normal BRCA1+/− women (mean age±SD of 41.5±8.8 years), from 14 triple negative sporadic breast cancers (ER-α-, PR-, HER2-) (men age of 57.8±13 years), and from 13 triple negative breast cancers from—BRCA1 mutated women (mean age of 49.3±10.7 years). The patients had signed up an informed consent (Comité de Protection des Personnes, biomedical project 11826) according to the Ethical National rules and processed according to the French law on clinical experimentation. The normal patients had no reported history of breast disease and pathological studies showed only normal breast tissue.

Tissues were analysed by immunohistochemistry (10% formalin fixed and paraffin embedded). Paraffin sections were then de-waxed and rehydrated and antigens sites were retrieved by treating sections in sodium citrate buffer overnight in the Retriever 2100 apparatus (PickCell Laboratories, The Netherlands). Sections were incubated with a primary polyclonal antibody against human total GR (1:75) (GR (H300), sc-8992, Santa Cruz, Heidelberg, Germany) or GR P-Ser211 (1:100) (#4161, Cell Signaling, Danvers, Mass., USA), and a streptavidin-biotin-peroxidase method (Vectastain kit, Abcys, Paris, France). A negative control (omitting the first antibody) and a positive control (normal appendix) were included in the series.

Cell Culture Procedures

MCF-7 and MDA-MB231 cell lines were respectively maintained in DMEM and in RPMI 1640 media (PAA Laboratories, Pasching, Austria) supplemented with 10% fetal calf serum. In normal human breast epithelial cells (HBE) experiments, cortisol (5 ng/ml) was added.

HBE cultures resulting from breast specimens, obtained from 12 of the 20 women without known BRCA mutation, with their informed consent according to the Ethical National rules and processed according to the French law on clinical experimentation. The patients had no reported history of breast disease and pathological studies showed only normal breast tissue. The procedure used for the HBE culture has been described elsewhere (Gompel et al, 1986). HBE cells were maintained in HAM F10 medium (PAA Laboratories, Pasching, Austria) containing NaHCO₃ (0.24%), penicillin-streptomycin (1%), cortisol (5 ng/ml), T3 (6.5 ng/ml), choleratoxin (10 ng/ml), transferrin (5 mg/ml), insulin (0.016 U/ml) and EGF (10 ng/ml) and 5% human serum.

Steroid Treatments

After seeding and transfection, cells were treated with DEX (100 nM) alone or in combination with AG (1 μM). Control cells were treated with ethanol as vehicle (1:1000). Treatments were carried out in a phenol red free medium containing 5% dextran-charcoal-stripped serum for a period of 24 h in MCF-7 and MDA-MB231 and 48 h in HBE cells.

BRCA1 siRNA Transient Transfections

HBE, MCF-7 and MDA-MB231 cells were transiently transfected with 10 nM of either siRNA control or siRNA BRCA1 using the HiPerFect Reagent (Qiagen, Courtaboeuf, France) in MCF7 and MDA-MB231, or the Lipofectamine LTX Reagent in HBE cells according to the manufacturer instructions (Invitrogen, Cergy-Pontoise France). SiRNA BRCA1 transfection consisted in a transfection of four siRNA BRCA1 sequences from Qiagen Flexitube Gene Solution GS672 (Hs_BRCA1_{—}9, Hs_BRCA1_{—}13, Hs_BRCA1_{—}14, Hs_BRCA1_{—}15) which recognised BRCA1 transcripts at 4 different positions. For gene or protein expression analyses, cells were washed 48 hours after siRNA and hormone treatments were performed for 24 h or 48 h in MCF-7 or MDA-MB231 and HBE cells respectively. For reporter transactivation experiments, cells were washed 48 hours after siRNA transfection and then transfected with the GRE-Luc or hGR 1A or 1B/C-Luc plasmid reporter using Lipofectamine Reagent for MCF-7 or MDA-MB231 cells or Lipofectamine LTX Reagent for HBE cells. 24 h after the reporter transfection and 72 h after the siRNA transfection, hormone treatments were performed for 24 h or 48 h in MCF-7 or MDA-MB231 and HBE cells respectively.

BRCA1 Vector and Transient Transfections

MCF-7 and MDA-MB231 cells were transiently transfected with 5 μg of either pcDNA3 empty vector or pcDNA3 expressing wt-BRCA1 using the Lipofectamine Reagent according to the manufacturer instructions (Invitrogen, Cergy-Pontoise France). For gene or protein expression analyses, cells were washed 24 hours after vector transfection and hormone treatments were performed for 24 h in MCF-7 or MDA-MB231. For reporter transactivation experiments, cells were washed 24 hours after vector transfection and then transfected with the GRE-Luc or hGR 1A or 1B/C-Luc plasmid reporter using Lipofectamine Reagent for MCF-7 or MDA-MB231 cells. 24 h after the reporter transfection and 48 h after vector transfection, hormone treatments were performed for 24 h in MCF-7 or MDA-MB231 cells.

Western Blots

Cell lysates (50 or 100 μg) were subjected to a combined lower 12% section and upper 6% section polyacrylamide gel electrophoresis and proteins were transferred and detected using a chemoluminescence procedure. BRCA1 expression was analyzed with the human anti-BRCA1 rabbit polyclonal primary antibody (1:500) (BRCA1 (C20), sc-642, Santa Cruz, Heidelberg, Germany). Total GR expression was analyzed with the human anti-GR rabbit polyclonal primary antibody (1:1000) (GR (H300), sc-8992, Santa Cruz, Heidelberg, Germany). GR P-Ser211 expression was analyzed with the human anti-GR PSer211 rabbit polyclonal primary antibody (1:1000) (GR P-Ser211 (#4161), Cell Signaling, Danvers, Mass., USA). Phosphorylated MAPK p38 expression was analyzed with the human anti-phospho-p38 (Thr 180/Tyr 182) rabbit polyclonal primary antibody (1:1000) (p-p38, sc-17852, Santa Cruz, Heidelberg, Germany). MAPK p38 expression was analyzed with the human anti-MAPK p38 rabbit polyclonal primary antibody (1:1000) (MAPK p38 (#9212), Cell Signaling, Danvers, Mass., USA). These five antibodies were then detected with an anti-rabbit HRP-conjugated secondary antibody (1:5000) (Santa Cruz, Heidelberg, Germany). Actin expression was analyzed with the human anti-β-actin mouse monoclonal primary antibody (1:20 000) (β-actin (AC-15); A5441, Sigma-Aldrich, St Quentin Fallavier, France), and then detected with an anti-mouse HRP-conjugated secondary antibody (1:2000) (Sigma-Aldrich, St Quentin Fallavier, France).

Reporter Enzyme Assays

Cells were transfected with GRE-Luc or hGR 1A or 1B/C-Luc plasmid reporters by using Lipofectamine Reagent or Lipofectamine LTX Reagent respectively in breast cell lines or HBE cells (Invitrogen, Cergy-Pontoise, France). Transfection was performed according to the manufacturer instructions. 24 h after transfection, breast cell lines and HBE cells were treated with hormones for 24 h or 48 h respectively. At the end of the experiment, cells were lysed and luciferase activity was determined using the Luciferase Assay System (Promega, Charbonniéres-les-bains, France).

Flow Cytometry Analysis

BRCA1 overexpression or silencing were performed in MCF-7 and MDA-MB231 cells. 24 h or 48 h after transfection, cells were treated with DEX (100 nM) alone or in combination with AG (1 μM). 24 h after treatment, cells were washed in PBS, trypsinized and centrifuged 5 minutes at 1350 rpm. Cells were fixed with 70% ethanol overnight and before analysis, cells were washed in PBS and stained with 10 μg/ml propidium iodide in PBS (containing 0.835 U/ml ribonuclease A). For each sample at least 10,000 cells were counted on a BD LSR II flow cytometer (BD Biosciences, Le Pont de Claix, France). After gating out doublets and debris, cycle distribution and subG1 phase were analyzed using the ModFit LT software (Verity Software House).

Real-Time Quantitative Reverse Transcription PCR (qRT-PCR)

Total RNA was extracted using TriZOL Reagent. 2 μg of total RNA was subjected to reverse transcription (RT) by using random primers for 1 h at 37° C. 2 μl of RT product was diluted (1:10) and subjected to quantitative PCR using sequence specific primers (300 nM) and Brillant SYBR GREEN QPCR master mix on an Mx3000P apparatus (Agilent Technologies, Massy, France). Thermocycling conditions: 1 cycle at 95° C. for 10 min and 40 cycles at 95° C. for 30 s, 60° C. for 1 min and 72° C. for 30 s. Gene expression values were normalized to the housekeeping gene 36B4.

Statistical Analysis

To determine the statistical significance of treatments, One-way ANOVA and Tukey-Kramer multiple comparisons tests were performed to compare the relative efficiency of each treatment with the Instat 3 software (GraphPad, USA). p<0.05 was considered as significant.

Results

Loss of GR P-Ser211 Expression in Normal and Tumoral Breast Tissues from BRCA1 Mutated and Non-Mutated Carriers.

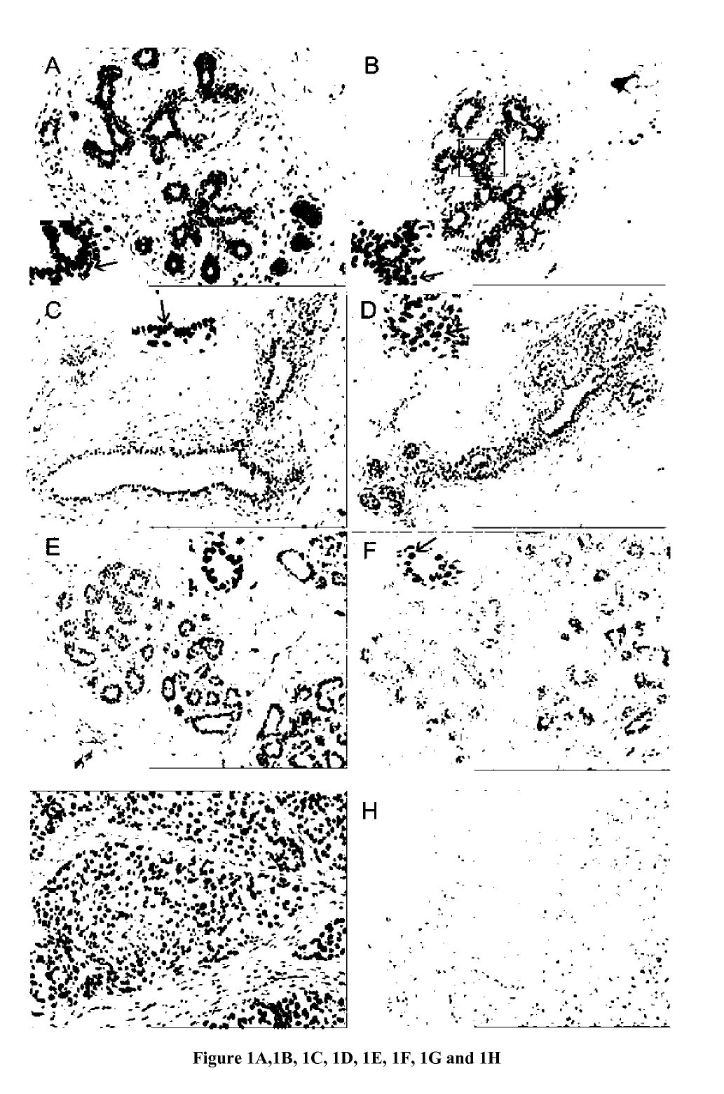

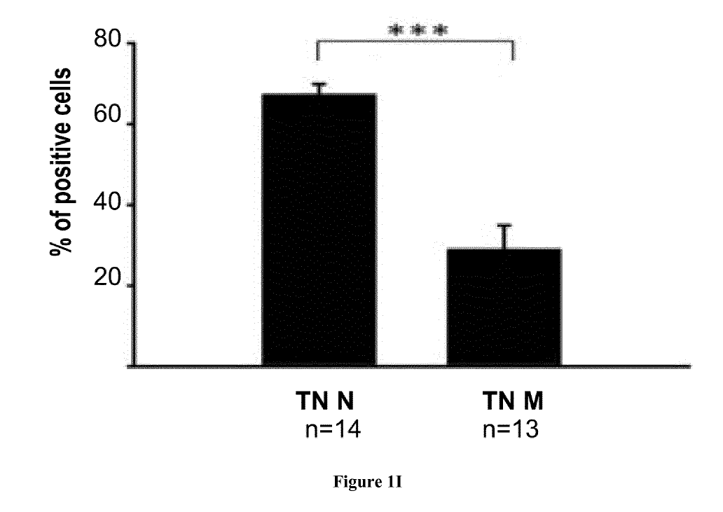

We first analyzed GR staining in normal breast tissues from 20 BRCA1 mutation carriers and 23 non-mutated women, who underwent prophylactic mastectomy or plastic surgery, respectively. GR staining was found more strongly expressed in the nuclei and the cytoplasm of myoepithelial cells than luminal cells and was identical in both series of breast tissues (FIG. 1A, 1B). Both antibodies used for total GR displayed a similar staining. In contrast, staining intensity of GR phosphorylated Ser 211 form (GR P-Ser211) was significantly more important in the nuclei of luminal cells. Furthermore, the staining was weaker in the nuclei of the myoepithelial and luminal cells from the mutation carriers than in the non-mutated breast tissues (FIG. 1C, 1D). Using the Allred score, we quantified the intensity and proportion of positive cells of the GR P-Ser211 in the two groups of women. The mean intensity score (±SEM) was 2.05±0.15 and median 2 for BRCA1^{+/-} versus 2.69±0.17 and median 3 for BRCA1^{+/+} (p<0.01).

Because the majority of breast cancers occurring in women with germline BRCA1 mutations are “triple negative” type (TN) (Sorlie et al. 2001; Sorlie et al. 2003), we also studied the expression of total GR and GR P-Ser211 in 13 TN type tumors from BRCA1 mutation carriers and in 14 TN tumors from non-mutated patients. Strikingly, GR P-Ser211 labeling from breast cancers associated with BRCA1 mutation was much weaker compared to sporadic breast cancers (FIG. 1E, 1F). In the BRCA1 associated tumors an average of 28.8%±6% of the cells expressed GR P-Ser211, whereas in sporadic breast cancers, a mean of 67.14%±2.7% of the cells expressed GR P-Ser211 (p<0.0001) (FIG. 1G). The staining intensity was also weaker; the score was 2.5±0.17 and 1.2±0.2 (p<0.001) for BRCA1^{+/+} and BRCA^{-/-} tumors, respectively.

The weaker GR P-Ser211 expression in healthy and tumoral tissues in BRCA1 mutation carrier suggested a contribution of BRCA1 on GR activation that is impaired in the mutated tissue. The following studies attempt to confirm this hypothesis.

BRCA1 Extinction Inhibits the Glucocorticoid Receptor Gene Regulation in HBE Cells

We first examined the effects of BRCA1 on glucocorticoid signaling pathway, when BRCA1 mRNA and protein contents were significantly abolished by BRCA1-specific siRNA (siBRCA1) in normal human primary breast cells (HBE cells). The transcription efficacy of Dexamethasone (DEX) was monitored on a reporter vector containing six GR-responsive elements, GRE-Luc. We observed that the level of GRE-Luc induction by DEX in presence of siBRCA1 was reduced by 50% compared to cells transfected with siControl (p<0.001), suggesting that BRCA1 expression level affects GR transcription gene activity. We confirmed this effect on specific glucocorticoid responsive genes. DEX induced upregulation of the Regulator of G-protein signaling (GOS8/RGS2) and the Mitogen-activated protein Kinase Phosphatase-1 (MKP-1) was strongly reduced when cells were transfected with siBRCA1 as compared to siControl (Wan and Nordeen 2002; Wu et al. 2005). In the same manner, the DEX induced down-regulation observed for the Immediate Early response gene X-1 (IEX-1) and tissue Plasminogen activator (tPa) was abolished in the presence of siBRCA1 as compared to siControl (Wan and Nordeen 2002; Wu et al. 2005). Supporting the interrelation between GR and BRCA1 in these cells, we observed a significant decrease of GR mRNA in the presence of siBRCA1.

These results illustrated that BRCA1 silencing drastically abrogated the DEX-mediated responses, indicating that BRCA1 is involved in GC dependent signaling pathways.

BRCA1 Status in Breast Cancer Cell Lines Regulates GC Dependent Proliferation and Apoptosis

Using BRCA1 silencing or overexpression approaches, we explored the contribution of BRCA1 expression on GC signaling pathway, in two breast cancer cell lines with differentiated and undifferentiated phenotypes; MCF-7 (ER⁺, PR⁺, GR⁺), and MDA-MB-231 (ER⁻, PR⁻ and GR⁺), respectively. As GCs are known to exert anti-proliferative and anti-apoptotic actions in breast cancer cell lines, we investigated the role of BRCA1 on cellular homeostasis and responses to GCs (Moran et al. 2000; Mikosz, et al. 2001; Mattern, et al. 2007).

DEX significantly decreased cell proliferation in MCF-7 and MDA-MB-231 cells, and this effect was abolished by treating cells with the anti-glucocorticoid compound (AG) ORG34116. This effect was not observed when cell lines were exposed to siBRCA1. BRCA1 overexpression enhanced the ability of DEX induced cell proliferation inhibition.

A significant decreased in cell apoptosis by DEX in MCF-7 and MDA-MB-231 cells was observed, as compared to controls. The anti-glucocorticoid AG also impeded this effect. Inhibition of BRCA1 expression abrogated DEX dependant anti-apoptotic effect whereas BRCA1 overexpression enhanced the DEX effect in both cell lines.

BRCA1 Status in Breast Cancer Cell Impacts GC Dependent Gene Expression

The transcription efficacy of DEX on GRE-Luc and endogenous GR target genes was studied in MCF-7 and MDA-MB-231 cells following BRCA1 silencing or overexpression. Interestingly, BRCA1 extinction reduced GRE-Luc constitutive activity by 2 fold and BRCA1 overexpression significantly enhanced the constitutive GRE-Luc activity in both cells. As expected, DEX induced a strong GRE-Luc transactivation in both cell types. This effect was drastically reduced by 4 to 6 fold in cells silenced for BRCA1 and enhanced in cells overexpressing BRCA1.

DEX stimulation of GC target genes GOS8, MKP-1, IEX-1 and tPa expression was altered accordingly to BRCA1 status in MCF-7 and MDA-MB-231 cells. The lack of BRCA1 induced an inhibition of GOS8 and MKP-1 DEX-mediated induction, and abrogated the DEX induced inhibition of IEX-1 and tPa mRNA expression.

In conditions of BRCA1 overexpression, we observed a mirror effect on gene induced by GCs. GS08 and MKP-1 mRNA DEX induced expressions were strikingly enhanced. However, no modification was observed on the mRNA levels of DEX repressed genes, IEX-1 and tPa.

These series of experiments confirmed that BRCA1 modulates GCs target gene activities as well as biological key functions mediated by GCs such as proliferation and apoptosis.

BRCA1 Regulates GR Expression and Activity

The potential mechanisms interconnecting BRCA1 and GR were investigated by analysis of GR expression modulation by BRCA1. In MCF-7 cells, GR mRNA was shown to be decreased in correlation with the efficiency of siBRCA1 to abolish BRCA1 expression over time. In MDA-MB-231 cells, GR mRNA also dramatically decreased in the presence of siBRCA1, as compared to siControl. BRCA1 stimulation of GR transcriptional activity was confirmed in both cell lines, by the abrogation of the DEX activation on the two main GR promoters (hGR 1A and hGR 1B) by siBRCA1 (Geng and Vedeckis 2004; Geng, et al. 2008). This abrogation of GR promoter transactivation by siBRCA1 was also observed in absence of DEX. The GR mRNA decrease was also correlated with a smaller quantity of total GR and GR P-Ser211 protein content in absence (Ø) as well as in presence of DEX. As expected, BRCA1 and GR mRNA were increased in MCF-7 and MDA-MB-231 cells when BRCA1 was overexpressed. GR promoter transactivations were also higher with BRCA1 overexpression and this mechanism occurred in the absence (Ø) or presence of DEX. These observations demonstrate that BRCA1 is also a key effector in GR transactivation of its own transcript in presence or absence of the ligand.

DEX promotes GR Ser211 phosphorylation. This DEX dependent induction of GR Ser211 phosphorylation was strongly blunted in conditions of siBRCA1 but not altered when BRCA1 was overexpressed. Constitutive phosphorylation of GR Ser211, upon BRCA1 overexpression, was observed in both cell lines non-stimulated by DEX. These results indicate that BRCA1 also participates in GR activity by augmenting its Ser211 phosphorylation rate in a ligand dependent and possibly independent manner.

The interaction between BRCA1 and various MAPK has been reported previously (Yan, et al. 2002). As MAPK p38 is involved in the GC induced phosphorylation of GR on Ser211 in lymphoid cells (Miller et al. 2005) and that BRCA1 induce MAPK p38 phosphorylation (Gilmore, et al. 2004), we hypothesized that BRCA1 could modulate GR-Ser211 level through the activation of MAPK p38. In our MCF-7 cellular model, the decrease of P-p38 expression by 2 fold in basal conditions when BRCA1 was silenced sustained this hypothesis. In addition, we used a MAPK phosphorylated p38 (P-p38) specific inactivator and stabilizer, the SB 202190 (SB). As expected, levels of P-p38 were increased in presence of SB, supporting the stabilization effect of SB on P-p38. Under basal conditions (Ø), in the absence of the inhibitor SB, GR P-Ser211 levels were either decreased or enhanced following BRCA1 silencing or overexpression, respectively. In cells exposed to SB, a constant decrease of GR P-Ser211 occurred, as compared to basal conditions, even in conditions of BRCA1 overexpression. This was also observed in cells treated by DEX+SB compared to DEX treatment alone. These results support the hypothesis for a role of P-p38 in GR Ser211 phosphorylation, and argue for the contribution of BRCA1 in the regulation of GR phosphorylation at the Ser211 by MAPK p38.

Our results establish a link between BRCA1 and GR expression and activation. Our data suggests that BRCA1 regulates GR activity through GR phosphorylation on Ser211, by modulating MAPK p38. GR will consequently need BRCA1 action to activate its auto-transcription and its target genes transcription. The alteration of BRCA1 expression impairs this pathway with both GR and GR activated forms of expression.

### DISCUSSION

In this study, we showed that wild type expressed BRCA1 is crucial for GCs to exert efficient signaling through GRSer211 phosphorylation. This evidence is provided by a set of data and observations coming from biopsies of normal human breast tissues from BRCA1 mutation carriers, triple negative breast cancers with documented mutations, and in vitro studies using BRCA1 cellular engineering. We conclude that in cells bearing mutated BRCA1, GR signaling pathway would be expected to be attenuated. These observations suggest a potential influence of GR deregulation in BRCA1 mutation carriers at two levels: the modulation of the risk to develop a breast cancer as well as an impact on cancer progression.

A DNA repair deficiency has been associated with BRCA1 haplo-insufficiency in mutated heterozygous cells (Rennstam, et al. 2010). That could promote the mammary cells to an early transformation process, and result in a higher risk of breast cancer development before 40-45 years for a BRCA1 mutation carrier. According to our results, this risk may also be modulated by a weaker GR activity in normal breast cells. At the physiological stage, GCs are continuously secreted by adrenal glands, and this release is enhanced under stress condition. Recently clinical and experimental studies concluded that stress contributes to cancer emergence and progression (Lillberg et al. 2003; Reiche et al. 2004). While chronic stress decreases breast cancer risk, it is known that acute stress enhances this risk (Vilasco, et al.; Kricker, et al. 2009; Michael, et al. 2009). In two recent studies, we showed that inhibition of GR dependent activity decreased the proliferative effect of DEX in normal cells, suggesting a protective effect against breast cancer in BRCA1^{+/-} cells (Courtin et al. 2011; Communal, et al. 2012). Thus, the relation observed between BRCA1 and GCs signalization pathway may have complex consequences on the risk of BRCA1^{+/-} cells transformation. This observation needs to be investigated in order to determine if a preventive treatment impacting GCs pathway could be consider.

In addition, the failure of GR activation may also influence breast carcinogenesis progression in BRCA1 mutated women. Here we showed that the BRCA1 invalidation in MCF-7 and MDA-MB-231 tumoral cells conducted to inhibit anti-proliferative but also anti-apoptotic effects of DEX. GCs are known to be powerful anti-inflammatory and immunosuppressive mediators (Baschant and Tuckermann 2010). The contribution of chronic inflammation in breast cancer progression has been extensively characterized. In patients with severe endometriosis, the chronic inflammatory state was correlated with an increased risk of breast and ovarian cancers with a ratio of 1.4 and 1.9, respectively (Brinton, et al. 1997). An elevated level of proliferating macrophages was associated with high grade, hormone receptor negative tumors, a basal-like subtype and subsequently a 77% increased risk of dying, in two independent cohorts of patients with breast cancer (Campbell, et al. 2011). The number of proliferating macrophages was consequently proposed as a significant predictor of recurrence and survival (Campbell et al. 2011). In addition, level of adaptive immunity key players, as high numbers of infiltrating activated B lymphocytes, and the activation and enhanced recruitment at the tumor site of specific subtype of T lymphocytes (CD4+), were also associated with breast cancer development and progression (Lee, et al. 1997; Kohrt, et al. 2005; DeNardo and Coussens 2007). Based on our results of an attenuated GC dependent response in BRCA1 mutated patients, weaker GCs anti-inflammatory activity should be observed in these patients and may lead to a reduced breast cancer protective effect. The crosstalk between GR levels and activity and BRCA1 as demonstrated by our work could constitute an important mechanism for breast cancer progression and needs to be further understood.

A relation between GR signalization pathway and BRCA1 was also previously described by Antonova et at (Antonova and Mueller 2008), who demonstrated that hydrocortisone was able to decrease BRCA1 expression in non malignant mouse mammary cells. In our experiments involving MCF-7 cells, down regulation of BRCA1 under DEX treatment was not detected. However, in conditions of GR inactivation, through p38 phosphorylation inhibition, an increase in BRCA1 level was observed suggesting that a crosstalk between GR and BRCA1 is also occurring in human breast cells.

GR signaling is strikingly regulated by MAPK cascade, on GR Ser211 and GR Ser203 phosphorylation by p38 and p42/44, respectively (Miller et al. 2005; Takabe, et al. 2008). The interaction between BRCA1 and various MAPK has been reported previously (Yan et al. 2002). BRCA1 is able to induce the phosphorylation of different kinases including the p38 activated MAPK (Gilmore et al. 2004). In this study, we showed that BRCA1 interferes on GR dependent gene activation and GR dependent cellular functions. Interestingly, the regulation was impaired on positively regulated genes but was not altered on negatively regulated genes in conditions of BRCA1 overexpression. This could be related to the different responsive sequence elements for GR mediated repression as recently reported (Surjit, et al. 2011). Our results, although preliminary, suggest that BRCA1 interferes with p38 activation, which is crucial for the stimulation of GR-dependent signaling through GR Ser211 activation, and therefore the abrogation of Ser211 phosphorylation is associated with GCs/GR pathway inhibition.

Accordingly, GR Ser211 phosphorylation staining in cancer patients bearing BRCA1 mutation could constitute a key diagnostic factor. The extension of this finding on sporadic cancers and the possible detection of lost BRCA1 expression or in BRCAness tumors, using a simpler method such as immunohistochemistry, would quickly facilitate the optimization of potential therapeutic strategies. The treatment using Poly (ADP-ribose) polymerase (PARP) inhibitors which have provided to be benefit on BRCA1 mutated breast cancer patient could be proposed to a larger set of patients (Javle and Curtin 2011).

In conclusion, using human normal and cancerous breast tissues, we established a mechanistic link between the multi-functional protein BRCA1 and GR signaling. Our results suggest that the lack of GRSer211 phosphorylation in tumors may be a potential diagnostic marker for a BRCA1 mutated carrier.

### REFERENCES

Throughout this application, various references describe the state of the art to which this invention pertains. The disclosures of these references are hereby incorporated by reference into the present disclosure.
Antoniou, A., P. D. Pharoah, et al. (2003). “Average risks of breast and ovarian cancer associated with BRCA1 or BRCA2 mutations detected in case Series unselected for family history: a combined analysis of 22 studies.” Am J Hum Genet 72(5): 1117-30.
Baschant, U. and J. Tuckermann “The role of the glucocorticoid receptor in inflammation and immunity.” J Steroid Biochem Mol Biol 120(2-3): 69-75.
Boulton, S. J. (2006). “Cellular functions of the BRCA tumour-suppressor proteins.” Biochem Soc Trans 34(Pt 5): 633-45.
Breslin, M. B., C. D. Geng, et al. (2001). “Multiple promoters exist in the human GR gene, one of which is activated by glucocorticoids.” Mol Endocrinol 15(8): 1381-95.
Burnstein, K. L., D. L. Bellingham, et al. (1991). “Autoregulation of glucocorticoid receptor gene expression.” Steroids 56(2): 52-8.
Campbell, M. J., N. Y. Tonlaar, et al. (2011). “Proliferating macrophages associated with high grade, hormone receptor negative breast cancer and poor clinical outcome.” Breast Cancer Res Treat 128(3): 703-11.
Courtin, A., L. Communal, et al. “Glucocorticoid receptor activity discriminates between progesterone and medroxyprogesterone acetate effects in breast cells.” Breast Cancer Res Treat.
Cramer, D. W. and O. J. Finn (2011). “Epidemiologic perspective on immune-surveillance in cancer.” Curr Opin Immunol 23(2): 265-71.
DeNardo, D. G. and L. M. Coussens (2007). “Inflammation and breast cancer. Balancing immune response: crosstalk between adaptive and innate immune cells during breast cancer progression.” Breast Cancer Res 9(4): 212.
Easton, D. F., D. Ford, et al. (1995). “Breast and ovarian cancer incidence in BRCA1-mutation carriers. Breast Cancer Linkage Consortium.” Am J Hum Genet 56(1): 265-71.
Ford, D., D. F. Easton, et al. (1998). “Genetic heterogeneity and penetrance analysis of the BRCA1 and BRCA2 genes in breast cancer families. The Breast Cancer Linkage Consortium.” Am J Hum Genet 62(3): 676-89.
Foulkes, W. D., I. M. Stefansson, et al. (2003). “Germline BRCA1 mutations and a basal epithelial phenotype in breast cancer.” J Natl Cancer Inst 95(19): 1482-5.
Frantz, B., T. Klatt, et al. (1998). “The activation state of p38 mitogen-activated protein kinase determines the efficiency of ATP competition for pyridinylimidazole inhibitor binding.” Biochemistry 37(39): 13846-53.
Galliher-Beckley, A. J. and J. A. Cidlowski (2009). “Emerging roles of glucocorticoid receptor phosphorylation in modulating glucocorticoid hormone action in health and disease.” IUBMB Life 61(10): 979-86.
Geng, C. D., J. R. Schwartz, et al. (2008). “A conserved molecular mechanism is responsible for the auto-up-regulation of glucocorticoid receptor gene promoters.” Mol Endocrinol 22(12): 2624-42.
Geng, C. D. and W. V. Vedeckis (2004). “Steroid-responsive sequences in the human glucocorticoid receptor gene 1A promoter.” Mol Endocrinol 18(4): 912-24.
Gilmore, P. M., N. McCabe, et al. (2004). “BRCA1 interacts with and is required for paclitaxel-induced activation of mitogen-activated protein kinase kinase kinase 3.” Cancer Res 64(12): 4148-54.
Govindan, M. V., F. Pothier, et al. (1991). “Human glucocorticoid receptor gene promotor-homologous down regulation.” J Steroid Biochem Mol Biol 40(1-3): 317-23.
Hermes, G. L., B. Delgado, et al. (2009). “Social isolation dysregulates endocrine and behavioral stress while increasing malignant burden of spontaneous mammary tumors.” Proc Natl Acad Sci USA 106(52): 22393-8.
Heximer, S. P., N. Watson, et al. (1997). “RGS2/GOS8 is a selective inhibitor of Gqalpha function.” Proc Natl Acad Sci USA 94(26): 14389-93.
Hinohara, K. and N. Gotoh (2010). “Inflammatory signaling pathways in self-renewing breast cancer stem cells.” Curr Opin Pharmacol 10(6): 650-4.
Hosey, A. M., J. J. Gorski, et al. (2007). “Molecular basis for estrogen receptor alpha deficiency in BRCA1-linked breast cancer.” J Natl Cancer Inst 99(22): 1683-94.
Jemal, A., R. Siegel, et al. “Cancer statistics, 2010.” CA Cancer J Clin 60(5): 277-300.
Katiyar, P., Y. Ma, et al. (2009). “Mechanism of BRCA1-mediated inhibition of progesterone receptor transcriptional activity.” Mol Endocrinol 23(8): 1135-46.
Kauff, N. D., J. M. Satagopan, et al. (2002). “Risk-reducing salpingo-oophorectomy in women with a BRCA1 or BRCA2 mutation.” N Engl J Med 346(21): 1609-15.
Kehrl, J. H. and S. Sinnarajah (2002). “RGS2: a multifunctional regulator of G-protein signaling.” Int J Biochem Cell Biol 34(5): 432-8.
Khan, J. A., L. Amazit, et al. “p38 and p42/44 MAPKs Differentially Regulate Progesterone Receptor A and B Isoform Stabilization.” Mol Endocrinol 25(10): 1710-24.
Kricker, A., M. Price, et al. (2009). “Effects of life event stress and social support on the odds of a > or =2 cm breast cancer.” Cancer Causes Control 20(4): 437-47.
Kruk, J. and H. Y. Aboul-Enein (2004). “Psychological stress and the risk of breast cancer: a case-control study.” Cancer Detect Prev 28(6): 399-408.
L. Antonova, C. R. M. (2008). “Hydrocortisone Down-Regulates the Tumor Suppressor Gene BRCA1 in Mammary Cells: A Possible Molecular Link Between Stress and Breast Cancer.” GENES, CHROMOSOMES & CANCER 47: 341-352.
Lange, C. A., T. Shen, et al. (2000). “Phosphorylation of human progesterone receptors at serine-294 by mitogen-activated protein kinase signals their degradation by the 26S proteasome.” Proc Natl Acad Sci USA 97(3): 1032-7.
Lillberg, K., P. K. Verkasalo, et al. (2003). “Stressful life events and risk of breast cancer in 10,808 women: a cohort study.” Am J Epidemiol 157(5): 415-23.
Ma, Y., P. Katiyar, et al. (2006). “The breast cancer susceptibility gene BRCA1 regulates progesterone receptor signaling in mammary epithelial cells.” Mol Endocrinol 20(1): 14-34.
Mattern, J., M. W. Buchler, et al. (2007). “Cell cycle arrest by glucocorticoids may protect normal tissue and solid tumors from cancer therapy.” Cancer Biol Ther 6(9): 1345-54.
Michael, Y. L., N. E. Carlson, et al. (2009). “Influence of stressors on breast cancer incidence in the Women's Health Initiative.” Health Psychol 28(2): 137-46.
Miki, Y., J. Swensen, et al. (1994). “A strong candidate for the breast and ovarian cancer susceptibility gene BRCA1.” Science 266(5182): 66-71.
Mikosz, C. A., D. R. Brickley, et al. (2001). “Glucocorticoid receptor-mediated protection from apoptosis is associated with induction of the serine/threonine survival kinase gene, sgk-1.” J Biol Chem 276(20): 16649-54.
Miller, A. L., M. S. Webb, et al. (2005). “p38 Mitogen-activated protein kinase (MAPK) is a key mediator in glucocorticoid-induced apoptosis of lymphoid cells: correlation between p38 MAPK activation and site-specific phosphorylation of the human glucocorticoid receptor at serine 211.” Mol Endocrinol 19(6): 1569-83.
Moran, T. J., S. Gray, et al. (2000). “The glucocorticoid receptor mediates a survival signal in human mammary epithelial cells.” Cancer Res 60(4): 867-72.
Pang, D., M. Kocherginsky, et al. (2006). “Dexamethasone decreases xenograft response to Paclitaxel through inhibition of tumor cell apoptosis.” Cancer Biol Ther 5(8): 933-40.
Park, J. J., R. A. Irvine, et al. (2000). “Breast cancer susceptibility gene 1 (BRCAI) is a coactivator of the androgen receptor.” Cancer Res 60(21): 5946-9.
Poole, A. J., Y. Li, et al. (2006). “Prevention of Brca1-mediated mammary tumorigenesis in mice by a progesterone antagonist.” Science 314(5804): 1467-70.
Qiu, M., A. Olsen, et al. (2003). “Mitogen-activated protein kinase regulates nuclear association of human progesterone receptors.” Mol Endocrinol 17(4): 628-42.
Razandi, M., A. Pedram, et al. (2004). “BRCA1 inhibits membrane estrogen and growth factor receptor signaling to cell proliferation in breast cancer.” Mol Cell Biol 24(13): 5900-13.
Rebbeck, T. R., H. T. Lynch, et al. (2002). “Prophylactic oophorectomy in carriers of BRCA1 or BRCA2 mutations.” N Engl J Med 346(21): 1616-22.
Reiche, E. M., S. O. Nunes, et al. (2004). “Stress, depression, the immune system, and cancer.” Lancet Oncol 5(10): 617-25.
Roberts, F. D., P. A. Newcomb, et al. (1996). “Self-reported stress and risk of breast cancer.” Cancer 77(6): 1089-93.
Shen, L., J. Guo, et al. (2006). “Distinct domains for anti- and pro-apoptotic activities of IEX-1.” J Biol Chem 281(22): 15304-11.
Shipp, L. E., J. V. Lee, et al. “Transcriptional regulation of human dual specificity protein phosphatase 1 (DUSP1) gene by glucocorticoids.” PLoS One 5(10): e13754.
Sorlie, T., C. M. Perou, et al. (2001). “Gene expression patterns of breast carcinomas distinguish tumor subclasses with clinical implications.” Proc Natl Acad Sci USA 98(19): 10869-74.
Sorlie, T., R. Tibshirani, et al. (2003). “Repeated observation of breast tumor subtypes in independent gene expression data sets.” Proc Natl Acad Sci USA 100(14): 8418-23.
Spillman, M. A. and A. M. Bowcock (1996). “BRCA1 and BRCA2 mRNA levels are coordinately elevated in human breast cancer cells in response to estrogen.” Oncogene 13(8): 1639-45.
Takabe, S., K. Mochizuki, et al. (2008). “De-phosphorylation of GR at Ser203 in nuclei associates with GR nuclear translocation and GLUTS gene expression in Caco-2 cells.” Arch Biochem Biophys 475(1): 1-6.
Tsirka, S. E., A. D. Rogove, et al. (1996). “Neuronal cell death and tPA.” Nature 384(6605): 123-4.
Vilasco, M., L. Communal, et al. “Glucocorticoid receptor and breast cancer.” Breast Cancer Res Treat 130(1): 1-10.
Wan, Y. and S. K. Nordeen (2002). “Overlapping but distinct gene regulation profiles by glucocorticoids and progestins in human breast cancer cells.” Mol Endocrinol 16(6): 1204-14.
Wu, M. X. (2003). “Roles of the stress-induced gene IEX-1 in regulation of cell death and oncogenesis.” Apoptosis 8(1): 11-8.
Wu, W., T. Pew, et al. (2005). “Glucocorticoid receptor-induced MAPK phosphatase-1 (MPK-1) expression inhibits paclitaxel-associated MAPK activation and contributes to breast cancer cell survival.” J Biol Chem 280(6): 4117-24.
Zhou, J. and J. A. Cidlowski (2005). “The human glucocorticoid receptor: one gene, multiple proteins and diverse responses.” Steroids 70(5-7): 407-17.

1. A method for screening a BRCA1 loss-of-function in a subject comprising the step consisting of determining the level of serine 211 phosphorylated glucocorticoid receptor (GR P-Ser211) expression in a tissue sample obtained from said subject.

2. The method according to claim 1 which comprises the step of comparing the level of GR P-Ser211 expression determined in the tissue sample with a reference value, wherein detecting differential in the level of expression determined in the tissue sample and the reference value is indicative that the subject has a BRCA1 loss-of-function.

3. A method for treating a cancer in a patient comprising i) screening a BRCA1 loss-of-function in the subject by the method according to claim 1 or 2 and ii) administering the patient with a PARP inhibitor when a BRCA1 loss-of-function is identified at step i).
